(19) United States
(12) Patent Application Publication
(10) Pub. No.: US 2012/0082719 A1
(43) Pub. Date: Apr. 5, 2012

(54) Compositions For Treating Chronic Viral Infections

(76) Inventor: Sam Poon Ang, Rowland Heights, CA (US)

(21) Appl. No.: 12/898,074

(22) Filed: Oct. 5, 2010

(51) Int. Cl. A61K 36/06; A61K 9/28; A61P 31/12; A61K 9/48

(52) U.S. Cl. 424/452; 424/195.15; 424/465

(57) ABSTRACT

The present invention describes dietary compositions and methods of using such compositions to treat chronic viral infections, including hepatitis B and hepatitis C infections.

### BACKGROUND OF THE INVENTION

1. Field of the Invention

The present invention relates generally to enhancing immune system function, and specifically to dietary compositions that enhance immune response, including methods of use thereof, for treating chronic viral infections.

2. Background Information

There are many chronic diseases in which infection persists because of a failure of the immune system to eliminate the infectious agent. Such diseases may be divided into at least two groups: those in which there is an obvious immune response that fails to eliminate the agent and those in which the infectious agent seems to be invisible to the immune system and barely induces a detectable response.

Regarding the first group, the immune response is often partly responsible for the pathogenic effects. For example, those agents which are associated with an aggressive TH₂-type response and may be characterized by high IgE levels (e.g., parasitic infections), damage is caused by a failure to completely eliminate the agent via an immune response. Another example would be mycobaterial infections, such as tuberculosis and leprosy, that result in persistent intracellular infection, where a TH₁-type response aids in containing the infection but also causes granuloma formation and tissue necrosis. Among viruses, hepatitis B and hepatitis C infections are commonly followed by persistent viral carriage and hepatic injury, resulting in death from hepatitis (i.e., cirrhosis or liver failure) or hepatoma (i.e., malignant hepatoma or hepatocellular carcinoma). With HIV, for example, very rarely are viral clearance and/or protection against subsequent re-infection observed.

For the second group of chronic infections, mostly viral, the immune response fails primarily because of the relative “tolerance” of the immune system to the agent. One example would be the herpes virus. As the virus is passed, it becomes latent in nerve tissue and may cause resultant lesions, which are frequently recurrent. This tolerance or “invisibility” seems to be caused by the viral protein ICP-47, which binds to TAP complex and inhibits peptide transport into the endoplasmic reticulum in infected cells. Thus, viral peptides are not presented to the immune system by MHC class I molecules.

Chronic infection with hepatitis B virus (HBV) or hepatitis C virus (HCV) is the leading cause of cirrhosis of the liver and hepatocellular carcinoma (HCC) in the world. It is estimated that 800,000 to 1.4 million Americans are chronically infected with HBV and 2.7 to 3.9 million Americans are chronically infected with HCV. However, these figures are believed to underestimate the true burden of disease in the United States. A more realistic estimate is that about 2 million Americans are chronically infected with HBV and that as many as 5 million are chronically infected with HCV. Many infected individuals are asymptomatic, and many—symptomatic or not—do not seek treatment.

Acute HBV or HCV infection can lead to acute liver failure in rare instances (<1%). However, a more significant problem is chronic infection, which can lead to cirrhosis in about 20% to 30% of individuals, and eventually to end-stage liver disease or HCC. Many individuals are unaware that they are infected until they develop signs or symptoms of cirrhosis or liver disease. Chronic HBV infection is responsible for 3,000 deaths per year in the United States, and chronic HCV infection is responsible for 12,000 deaths per year. Chronic infection can thus have dire consequences: debilitating symptoms, impaired quality of life, disability, costly health care expenditures, and death.

Worldwide it is estimated that about 3% of the world's population is infected with HCV. No vaccine for hepatitis C is presently available, and the current treatment modalities (e.g., pegylated interferon and ribavirin) are only effective in about 30-60% of the population. Further, such modalities have potential serious side effects, including but not limited to, seizures, acute heart and kidney failure, and anemia.

As such, what is needed is a safe and efficacious modality that is not associated with side effects of the present therapeutic regimes.

### SUMMARY OF THE INVENTION

The present invention describes dietary compositions and methods of using such compositions to treat chronic viral infections.

In one embodiment, a dietary and/or therapeutic supplement composition is disclosed including, in admixture, alkylglycerols, a sesquiterpene lactone, agaricus bisporous or an extract thereof, and chlorophyllin. In one aspect, the supplement composition is formed by adding alkylglycerols to a composition comprising a sesquiterpene lactone, agaricus bisporous or an extract thereof, and chlorophyllin in situ or situs, and where the sesquiterpene lactone is artemisinin. In a related aspect, the composition including the sesquiterpene lactone, agaricus bisporous or an extract thereof, and chlorophyllin, further comprises one or more ingredients including idebenone, melatonin, milk thistle, at least 2 separate gluconic acid salts, at least 2 separate fat soluble vitamins, and/or combinations thereof. In a further related aspect, the gluconic acid salts are zinc and copper. In another related aspect, the fat soluble vitamins are vitamin D3 and retinyl acetate.

In another aspect, the composition includes about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

In one aspect, the admixture comprises about 600 to 1200 mg of alkylglycerols, about 600 to 1500 mg of sesquiterpene lactone, about 600 to 1000 mg of agaricus bisporous or an extract thereof, and about 300 mg of chlorophyllin. In another aspect, the composition further includes at least one pharmaceutically acceptable excipient.

In one aspect, the composition is formulated in an enteric coated capsule. In another aspect, the composition is formulated in an enteric coated tablet.

In another aspect, the composition includes about 600 to 1200 mg of alkylglycerols, about 600 to 1500 mg of sesquiterpene lactone, about 600 to 1000 mg of agaricus bisporous or an extract thereof, about 300 mg of chlorophyllin, about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

In another embodiment, a kit is disclosed including a first container comprising an alkylglycerol containing composition, a second container comprising an artemisinin, agaricus bisporous or extract thereof, and chlorophyllin containing composition, instructions including procedures for mixing the compositions of the first and second container, and a label.

In one aspect, the instructions further include procedures for administering an admixture including the compositions of the first and second container to a subject in need thereof. In another aspect, the second container further includes one or more ingredients including idebenone, melatonin, milk thistle, at least 2 separate gluconic acid salts, at least 2 separate fat soluble vitamins, and/or combinations thereof In a related aspect, the gluconic acid salts are zinc and copper. In another related aspect, the fat soluble vitamins are vitamin D3 and retinyl acetate.

In one aspect, the kit includes about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

In another aspect, the admixture comprises about 600 to 1200 mg of alkylglycerols, about 600 to 1500 mg of artemisinin, about 600 to 1000 mg of agaricus bisporous or extract thereof, and about 300 mg of chlorophyllin.

In one aspect, the compositions of the first and second container further include at least one pharmaceutically acceptable excipient. In a related aspect, the composition including artemisinin, agaricus bisporous or extract thereof, and chlorophyllin is formulated in an enteric coated capsule. In another related aspect, the composition including artemisinin, agaricus bisporous or extract thereof, and chlorophyllin is formulated in an enteric coated tablet.

In another aspect, the kit includes about 600 to 1200 mg of alkylglycerols, about 600 to 1500 mg of artemisinin, about 600 to 1000 mg of agaricus bisporous or extract thereof, about 300 mg of chlorophyllin, about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

In one embodiment, a method of supplementing the diet of a subject is disclosed which includes the step of administering to the subject a dietary supplement including alkylglycerols, a sesquiterpene lactone, agaricus bisporous or an extract thereof, and chlorophyllin.

In one aspect, the subject is infected with a virus. In a related aspect, the virus is includes Hepatitis B, Hepatitis C, Epstein Barr Virus, HIV, Cytomegalovirus, Herpes Simplex Virus 1, Herpes Simplex Virus 2, Human Papillomavirus, Adenovirus, Kaposi's Sarcoma-Associated Herpesvirus, Torquetenovirus, JC Virus, and BK Virus.

In another aspect, the dietary supplement is given via 3 equally divided daily doses. In a related aspect, the dietary supplement administered includes about 600 to 1200 mg of alkylglycerols, about 600 to 1500 mg of artemisinin, about 600 to 1000 mg of agaricus bisporous or extract thereof, about 300 mg of chlorophyllin, about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate. In another aspect, administration of the dietary supplement reduces viral load.

### BRIEF DESCRIPTION OF THE DRAWINGS

FIG. 1 illustrates the activation of the immune system by antigen.

FIG. 2 shows the chemical structure of alkylglycerol consisting of a backbone of glycerol attached with an ether link to an alkyl group.

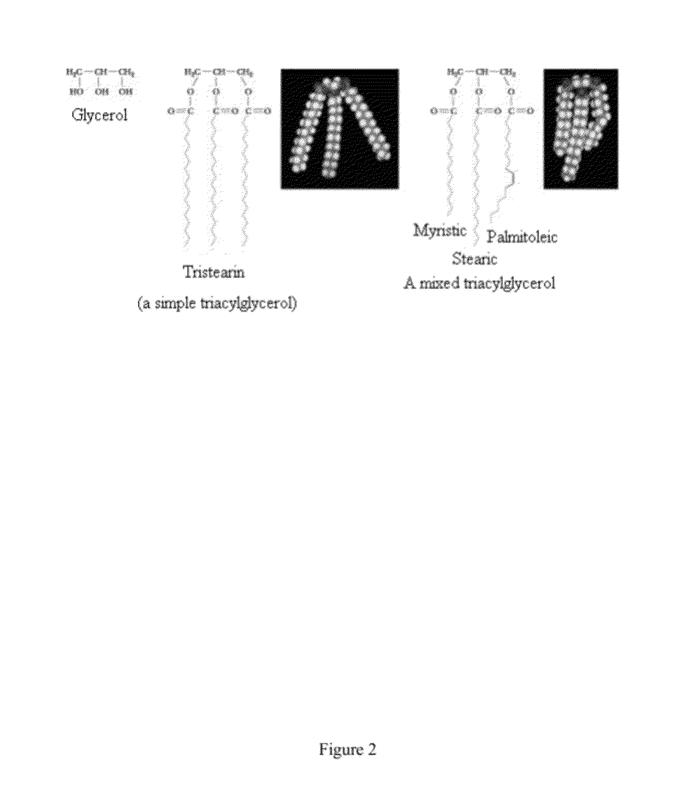

### DETAILED DESCRIPTION OF THE INVENTION

Before the present compositions, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a composition” includes one or more compositions, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “supplement” includes, but is not limited to, an herb, enzyme, vitamin, animal oil, naturally occurring hormone, medicinal plant, animal extract, or diet-oriented compound or medicament such as those traditionally used in the treatment of a disease or a medical condition.

As used herein, “in situ” or “in situs” means restricted to a site within the body of a subject.

As used herein, “therapeutic,” including grammatical variations thereof, means to provide treatment for a disease or medical condition. For example, the composition as described herein improves at least one parameter associated with a disease or medical condition (e.g., chronic infection). Further, the dietary supplement as disclosed reduces viral load (e.g., by about 40% to 50%, about 50% to 60%, about 60% to 70%, about 70% to 80%, about 80% to 90%, about 90% to 100% or reduces viral load to the detection limit of PCR and/or immunoassay) or clears detectable virus from the subject when administered. In a related aspect, reduction in viral load may be assayed by any technique known in the art (e g , immunologically or by nucleic acid-based assay, e.g., PCR).

As used herein, “admixture,” including grammatical variations thereof, means an additional ingredient that is added by mixing one component or composition with a separate component or composition. For example, the alkylglycerols of the present invention may be mixed separately with 10 other components as recited herein (e.g., artemisinin, agaricus bisporous or extract thereof, chlorophyllin, idebenone, melatonin, milk thistle, at least 2 separate gluconic acid salts, at least 2 separate fat soluble vitamins, and/or combinations thereof).

As used herein, “subject,” including grammatical variations thereof, means a human or an animal that is exposed to experimental, therapeutic other observational procedures.

As used herein, “infectious agent,” including grammatical variations thereof, means any power, principle or substance by which something is accomplished, or which is capable of producing a chemical, physical or biological effect such as a disease. For example, such agents include, but are not limited to, Hepatitis B, Hepatitis C, Epstein Barr Virus, HIV, Cytomegalovirus, Herpes Simplex Virus 1, Herpes Simplex Virus 2, Human Papillomavirus, Adenovirus, Kaposi's Sarcoma-Associated Herpesvirus, Hepatitis B, Torquetenovirus, JC Virus, and BK Virus.

Currently, there are two main immunological approaches to the treatment of chronic infection. One is to try to boost or change the pattern of the host immune response using, e.g., cytokine therapy. The second is to attempt therapeutic vaccination to see whether the host immune response can be “supercharged” by immunization with antigens from the infectious agent in combination with adjuvant. As stated above, no vaccine for hepatitis C is presently available, and the current treatment modalities (e.g., pegylated interferon and ribavirin), which are only effective in about 30-60% of the population, have potential serious side effects.

The present invention discloses a specific mix of supplements, which are free of serious side effects, to turn on “switches” of the immune system. In one aspect, the admixture as presently disclosed is able to reverse Hepatitis C virus infections in 30 days and Hepatitis B virus infections in 23 days. Because all human immune systems behave in a relatively predictable fashion, except in rare cases of in-born T cell defects, the protocol should be 100% effective, regardless of any type or genotype of virus or subject (e.g., should work with those individuals with multiple gene morphology related to glutathione-S-transferase alleles; see, e.g., Kandemir et al., Hepatogastroenterology (2008) 55(86-87):1729-1733). Further, the infectious agent is not likely to mutate against the treatment due to somatic hyper-mutation of the antibodies.

The formulation of the present application provides a way to induce the immune system with the right mixture of nutrition so it can produce potent antibodies against chronic virus infections such as Hepatitis B, Hepatitis C, Herpes Virus, Epstein Barr Virus, and the like. In one aspect, the formulation of the present invention may be used to treat chronic bacterial infections such as chronic bacterial prostatitis, lyme diseases, leprosy, and the like.

It is known in the art that chronic virus infections such as Herpes, Hepatitis B and Hepatitis C infections can be controlled by anti-virus drugs, however, there is no evidence that such chronic infections are cured. Nevertheless, it is of interest to note that up to 90% of adults who contracted hepatitis B virus develop immunity without treatment, and only 5% become chronic carriers of the B virus. As for Hepatitis C, about 20% of patients develop immunity against the C virus, while 80% of patients become life-long Hepatitis C victims.

While not being bound by theory, many immunologists think the humoral (antibody) response is weak or ineffective, which is why patients develop antibodies against Hepatitis C, HIV, CMV, EBV and Herpes viruses yet are not immune to it. Therefore, most current research efforts focus on cell mediated response and/or anti-virus replication approaches.

There is a common belief among immunologists and virologists that the body's immune system preferentially utilizes immunological memory based on a previous infection when a second slightly different version of that foreign entity (e.g. a virus or bacterium) is encountered. This leaves the immune system “trapped” by the first response it has made to each antigen, unable to mount potentially more effective responses during subsequent infections. This condition has been denoted as the “Original Antigenic Sins.” This theory, proposed by Thomas Francis in 1960, conveniently explained why people developed chronic virus infections such as Hepatitis B, C, HIV, Herpes, and HPV, but it was never clinically proven.

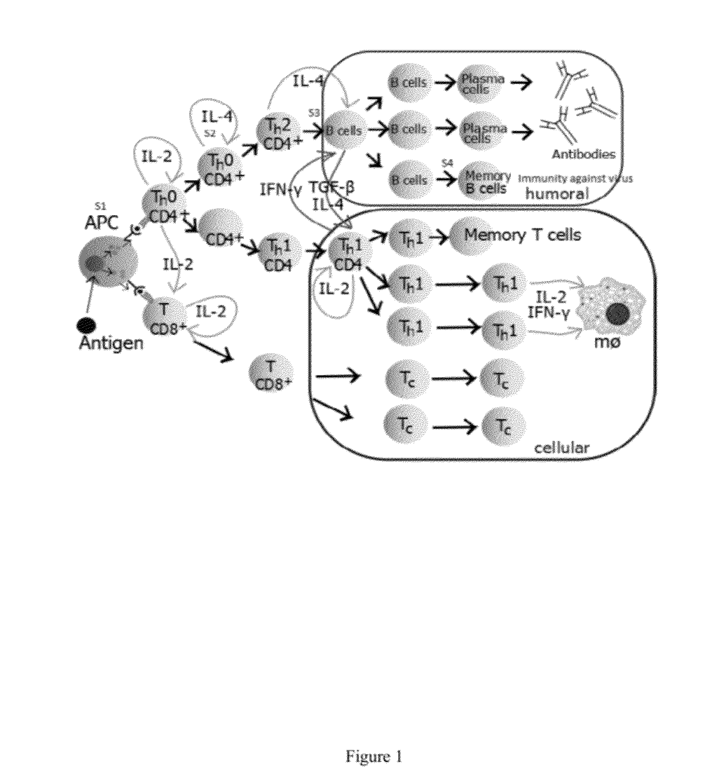

The human immune system relies on antigen processing cells (APCs) to recognize invading microbial such as virus and bacteria in order to develop antibodies to destroy them (see, e.g., FIG. 1). There are three types of APCs: B cells, macrophages and dendritic cells (DCs). Both B cells and macrophages are permanent cells, meaning that most of the time, the human immune system depends on them as the main APCs to detect invading viruses and bacteria. Unfortunately, both B cells and macrophages are very poor quality APCs. This may explain why people develop chronic virus and bacterial infections; in other words, while not being bound by theory, if immune systems depend on B cells and macrophages as APCs, they are less likely to develop immunity against the invader, simply because the antibody response induced by B cells and macrophages is just not precise enough to destroy the invaders.

Dendritic cells were first described by Paul Langerhans in the late nineteenth century. It wasn't until 1973, however, that the term “dendritic cells” was coined by Ralph M. Steinman and Zanvil A. Cohn. Some of the basic functions of DCs were first reported by Ralph Steinman in 1980, but today not enough has been learned about these cells that only appear in the body under certain conditions and for only about 3 days at a time. DCs are the most powerful APCs because they are the only cells that are able to induce primary immune responses, thus permitting establishment of immunological memory. In fact, it is estimated that a DC to T cell ratio of 1 to 100 is all the body needs to initiate vigorous and optimal immune responses.

DCs select the rare specific T cells from the assembled repertoire that recognize the specific peptide information the DCs are carrying. However, only one in 10,000-100,000 of the T cells in that repertoire are able to respond to this information. In comparison with B cells and macrophages, DCs are 10,000 to 100,000 times more precise in selecting the most suitable T cell to initiate the antibody response. In other words, when compare with B cells and macrophages, DCs are 10,000 to 100,000 more precise in recognizing the invaders.

MHC (major histocompatibility complex) Class II molecules are found only on macrophages, dendritic cells and B cells. MHC Class II molecules interact exclusively with CD4+ (“helper”) T cells (again, see FIG. 1). The helper T cells then help to trigger an appropriate immune response which may include localized inflammation and swelling due to recruitment of phagocytes or may lead to a full-force antibody immune response due to activation of B cells. Mature DCs are the only cell group that expresses MHC Class II at all times. MHC products and MHC peptide complexes are 10 to 100 folds higher on DCs than B cells and macrophages. Thus, DCs are 10 to 100 times more efficient in activating an antibody response than B cells and macrophage cells. In fact, it is now art recognized that immunity through vaccination will not take place without DCs.

One problem in exploiting DCs for immunotherapy is that they are temporary cells, meaning the body needs specific conditions to trigger the release of the DCs. While not being bound by theory, it may be that those who fail to develop immunity against invading microbial must also have failed to produce sufficient DCs, and therefore, their immune system is unable to recognize the infective agent effectively.

The stimulation of DCs may represent a vital first step in the development of potent antibodies against any invading agent, viral or microbial. However, the immune system needs more help than a simple boost in DCs in order to produce potent antibodies against invading virus and bacteria. According to the present invention, the human immune system contains four “switches.” While not being bound by theory, if all four switches are turned on in cascading sequence, the immune system will develop potent antibodies against any infectious agent, including Hepatitis B, Hepatitis C, HIV, Herpes, HPV virus and Epstein Barr virus and the like. This suggests that the immune system needs a very specific set of enhancers (e.g., nutrients) to produce potent antibodies in order to defend itself against infectious agents. In other words, in those who developed immunity against, for example, Hepatitis B or C virus infections, these requirements have been met. However, for those who suffer from chronic virus infections, such switches have not been turned on, as the specific requirements have not been met. Again, while not being bound by theory, the formulation of the present invention seems to “turn-on” these switches.

In one embodiment, a dietary and/or therapeutic supplement composition for the treatment of infectious agents is disclosed including, in admixture, alkylglycerols, a sesquiterpene lactone, agaricus bisporous or an extract thereof, and chlorophyllin. In one aspect, the supplement composition is formed by adding alkylglycerols to a composition comprising a sesquiterpene lactone, agaricus bisporous or an extract thereof, and chlorophyllin in situ or situs, and where the sesquiterpene lactone is artemisinin.

The liver oil from the rat fish and other elasmobranch fish is particularly rich in alkylglycerols. In natural sources they are always found esterified with fatty acids.

The ratfish (Chimera monstrosa), like the dog fish and the shark, is a member of the elasmobranch class of fish which are characterized by their distinctive lamellate gills. The oil from the liver of the fish has been used for centuries to treat lymphodenopathy. The substances in the oil that have accounted for these medicinal properties are the alkylglycerols.

The glycerol esters are widely distributed in animal tissues, although the liver of the elasmobranch fish remains the richest natural source.

Shark oil may be obtained commercially (see, e.g., ALKYROL, Eurohealth, Inc., Parkside, Pa.). Common fatty alcohols found in shark liver oil are chimyl alcohol, batyl alcohol and selachyl alcohol. Main ingredients of commercial sources may contain squalamine, 20% alkylglycerols, squalene, omega-3 oils, free fatty acids, vitamin E, Fe, Zn, and Cu. Synthetic alkylglycerols have been generated and shown to possess adjuvant properties (see, e.g., Acevado et al., Vaccine (2006) 24(Supp. 2):532-533; see, also, U.S. Pat. No. 4,173,641), as well as anticancer properties.

Alkylglycerols are potent macrophage stimulating agents. Administration of small amounts of alkylglycerols to mice greatly enhances macrophage activation for Fc mediated ingestion activity at the 5th day post treatment (Yamamoto et al., Can Res (1998) 48:6044-649).

It is known in the art that plants can convert alkylglycerols into glycerolphospholipid intermediates which in-turn have anti-viral activities (see, e.g., Mangold et al., Lipids (1991) 26(12):1086-1092).

The present invention discloses the use of alkylglycerols in combination with other ingredients as recited herein as an effective admixture for treating viral infection. As disclosed herein, the alkylglycerols may be administered at about 100 to 200 mg, about 300 to 600 mg, about 600 to 1200 mg, about 1200 to 1500 mg and about 1500 to 2000 mg of alkylglycerols.

Artemisinin, also known as qinghaosu, and its derivatives are a group of drugs that possess the most rapid action of all current drugs against falciparium malaria.

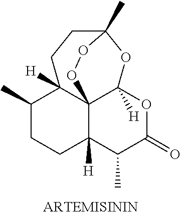

Treatments containing an artemisinin derivative (artemisinin-combination therapies, ACTs) are now standard treatment worldwide for falciparum malaria. The starting compound, artemisinin (a sesquiterpene lactone), is isolated from the plant Artemisia annua, a herb described in Chinese traditional medicine, though it is usually chemically modified and combined with other medications.

Use of the drug by itself as a monotherapy is explicitly discouraged by the WHO as there have been signs that malarial parasites are developing resistance to the drug. Combination therapies that include artemisinin are the preferred treatment for malaria and are both effective and well tolerated in patients. The drug is also increasingly being used in vivax malaria as well as being a topic of research in cancer treatment.

The present invention discloses the use of artemisinin in combination with alkylglycerols and other ingredients as recited herein as an effective admixture for treating viral infection. As disclosed herein, the artemisinin may be administered at about 100 to 600 mg, about to 600 to 1500 mg, or about 1500 to 2000 mg of artemisinin.

Agaricus bisporus—known variously as the common mushroom, button mushroom, white mushroom, table mushroom, cremini, Champignon mushroom, crimini mushroom, Swiss brown mushrooms, Roman brown mushrooms, Italian brown, Italian mushroom, or cultivated mushroom—is an edible basidiomycete mushroom native to grasslands in Europe and North America. Agaricus bisporus is cultivated in more than 70 countries and is one of the most commonly and widely consumed mushrooms in the world.

These mushrooms can contain high amounts of vitamin D especially when UV-irradiated. Agaricus bisporus also contains sodium, potassium, and phosphorous, conjugated linoleic acid, and antioxidants. In addition, the table mushroom has been shown to possess possible immune system enhancing properties. An in vitro study demonstrated the mushroom enhanced dendritic cell function (see, e.g., Ren et al., J Nutr (2008) 128(3):544-550).

The present invention discloses the use of Agaricus bisporus in combination with alkylglycerols, artemisinin, and other ingredients as recited herein as an effective admixture for treating viral infection. As disclosed herein, the Agaricus bisporus (or extract thereof) may be administered at about 100 to 600 mg, about to 600 to 1000 mg, or about 1000 to 2000 mg of Agaricus bisporus.

Chlorophyllin refers to any one of a group of closely related water soluble salts that are semi synthetic derivatives of chlorophyll, differing in the identity of the cations associated with the anion. Its most common form is a sodium/copper derivative used as a food additive and in alternative medicine. Chlorophyllin is the active ingredient in a number of internally-taken preparations intended to reduce odors associated with incontinence, colostomies and similar procedures, as well as body odor in general. It is also available as a topical preparation, purportedly useful for both treatment and odor control of wounds, injuries, and other skin conditions. Chlorophyllin is extracted from green plants, nettle, spinach or grass.

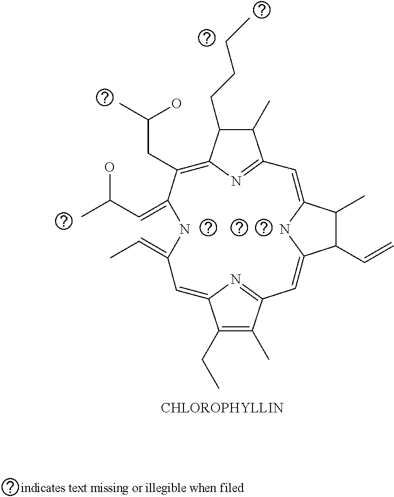

The present invention discloses the use of chlorophyllin in combination with alkylglycerols, artemisinin, Agaricus bisporus and other ingredients as recited herein as an effective admixture for treating viral infection. As disclosed herein, chlorophyllin may be administered at about 100 mg, about 200 mg, about 300 mg, about 400 mg, about 500 mg or about 600 mg of chlorophyllin.

In one embodiment, a composition is disclosed including alkylglycerols in admixture with a sesquiterpene lactone (e.g., artemisinin), agaricus bisporous (or an extract thereof), and chlorophyllin. In one aspect, the admixture essentially includes about 600 to 1200 mg of alkylglycerols, about 600 to 1500 mg of artemisinin, about 600 to 1000 mg of agaricus bisporous or extract thereof, and about 300 mg of chlorophyllin.

In addition to the above components, the admixture may contain idebenone, melatonin, milk thistle, at least 2 separate gluconic acid salts, at least 2 separate fat soluble vitamins, and/or combinations thereof. In a related aspect, the gluconic acid salts are zinc and copper.

Idebenone (e.g., trade names CATENA and SOVRIMA) is an experimental drug, initially developed for the treatment of Alzheimer's disease and other cognitive defects. This has been met with limited success. Recently it has started to be investigated for the treatment of neuromuscular diseases, including Friedreich's ataxia and Duchenne muscular dystrophy. Chemically, idebenone is an organic compound of the quinone family. It is also promoted commercially as a synthetic analog of coenzyme Q10 (CoQ10), including that dietary supplements containing idebenone may be used as a potent version of CoQ10.

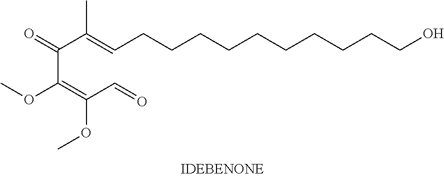

Milk thistle (Silybum marianum) is a flowering plant of the daisy family (Asteraceae). They are native to the Mediterranean regions of Europe, North Africa and the Middle East. The name “milk thistle” derives from two features of the leaves: they are mottled with splashes of white and they contain a milky sap.

The seeds of the milk thistle have been used for over 2000 years to treat chronic liver disease and protect the liver against toxins. Increasing research is being undertaken on the physiological effects, therapeutic properties and possible medical uses of milk thistle. The active ingredient (silymarin) is made of flavonolignans (most commonly silybin). Silymarin undergoes enterohepatic recirculation which results in higher concentration in the liver cells rather than in the liver. Considered an anti-inflammatory, milk thistle increases lymphocyte proliferation, INF gamma, IL-4, and IL-10. In the U.S., milk thistle is used to treat viral infection. However, evidence to date is inconsistent or limited (see, e.g., Rainone, Am Family Physician (2005) 72(7):1285-1288). In one aspect, the admixture of the present invention further includes about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

For oral administration, the components of the composition as disclosed herein may be incorporated into a tablet, aqueous or oil suspension, dispersible powder or granule, microbead, emulsion, hard or soft capsule, syrup or elixir. The components of the composition may also be administered separately, for example, alkylglycerols are separately mixed with a composition including a sesquiterpene lactone, agaricus bisporous or an extract thereof, and chlorophyllin. Compositions may be prepared according to any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic pharmaceutically acceptable excipients suitable for tablet manufacture are acceptable.

The phrases “pharmaceutically and/or pharmacologically acceptable” refer to molecular entities and/or compositions that do not produce an adverse, allergic and/or other untoward reaction when administered to a subject as appropriate. In addition, “pharmaceutically acceptable” means that the agent should be acceptable in the sense of being compatible with the other ingredients of the formulation. Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

Another embodiment of the present invention is a pharmaceutical composition comprising an enteric-coated form of the compositions. Any pharmaceutical formulation well known in the art can be coated with an enteric coating. In one embodiment, the formulation is a tablet, capsule or microbead.

The enteric coating may prevent dissolution of the tablet, capsule or microbead in the acidic environment of the stomach. Instead, this coating dissolves in the small intestine at a more neutral pH. Such enteric coated compositions are described by Bauer et al., Coated Pharmaceutical Dosage Forms. Fundamentals, Manufacturing Techniques, Biopharmaceutical Aspects, Test Methods and Raw Materials, CRC Press, Washington, D.C., 1998, the entire contents of which are hereby incorporated by reference.

Formulations for oral use may also be presented as hard gelatin capsules wherein the active ingredient(s) is mixed with an inert solid diluent, for example calcium carbonate, calcium phosphate or kaolin, or as soft gelatin capsules wherein the active ingredient(s) is mixed with water or an oil medium, such as peanut oil, liquid paraffin or olive oil.

Aqueous compositions may be used in the present invention and comprise an effective amount of a therapeutic chemical compound or pharmaceutically acceptable salts thereof dissolved and/or dispersed in a pharmaceutically acceptable carrier and/or aqueous medium.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For administration, preparations may meet sterility, pyrogenicity, general safety and/or purity standards as required by FDA Office of Biologics standards.

The biological material may be extensively dialyzed to remove undesired small molecular weight molecules and/or lyophilized for more ready formulation into a desired vehicle, where appropriate. The preparation of an aqueous compositions that contain an effective amount of a therapeutic agent as an active component and/or ingredient will be known to those of skill in the art in light of the present disclosure. Typically, such compositions can be prepared as liquid solutions and/or suspensions; solid forms suitable for using to prepare solutions and/or suspensions upon the addition of a liquid prior to administration can also be prepared; and/or the preparations can also be emulsified.

The pharmaceutical forms suitable for use include sterile aqueous solutions and/or dispersions; formulations including sesame oil, peanut oil and/or aqueous propylene glycol; and/or sterile powders for the extemporaneous preparation of sterile solutions and/or dispersions. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

Therapeutic agents of the present invention can be formulated into a composition in a neutral and/or salt form. Pharmaceutically acceptable salts, include the acid addition salts and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In some cases, it will be preferable to include isotonic agents, for example, sugars and/or sodium chloride. Prolonged absorption of the compositions can be brought about by the use in the compositions of agents delaying absorption, for example, aluminum monostearate and/or gelatin.

Sterile solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile solutions, the preferred methods of preparation are vacuum-drying and/or freeze-drying techniques which yield a powder of the active ingredient plus any additional desired ingredient from a previously sterile-filtered solution thereof

Upon formulation, solutions will be administered in a manner compatible with the dosage formulation and/or in such amount as is therapeutically effective. The formulations are easily administered in a variety of dosage forms, such as the type of solutions described above.

Therapeutic kits of the present invention are kits comprising a therapeutic agent such those components as disclosed. Such kits will generally contain, in suitable container means, a pharmaceutically acceptable formulation of a therapeutic agent(s). The kit may have a single container means, and/or it may have distinct container means for each compound. The kit may also contain instruction for administration/mixing the components therein. The instructions may be a pamphlet, CD, or other computer readable medium. Further, the instructions may provide information about a website which may contain downloadable content.

The following examples are intended to illustrate but not limit the invention.

### EXAMPLES

### Example 1

### Preparation of the Dietary Supplement

The formulation consists of the 11 ingredients in two parts:

Part one consists of 600 mg to 1200 mg of alkylglycerols in 3 dividing doses daily. Alkylglycerols are extracted from shark liver oil, and should not be mixed with the rest of the 10 ingredients. For the purposes of the present examples, the alkylglycerols are delivered in the form of shark liver oil (softgel), where each 1000 mg of shark oil contains 200 mg of alkylglycerols.

Part two consists of the following 10 ingredients in an enteric coated capsule or tablet to ensure that their potency is not affected by stomach acid.
a. Artemisinin 600 mg to 1500 mg in 3 dividing doses daily
b. Idebenone 300 mg in 3 dividing doses daily
c. Agaricus bisporus 600 mg to 1000 mg in 3 dividing doses daily
d. Chlorophyllin 300 mg in 3 dividing doses daily
e. Melatonin 30 mg in 3 dividing doses daily
f. Milk Thistle 700 mg in 3 dividing doses daily
g. Zinc Gluconate 30 mg in 3 dividing doses daily
h. Copper Gluconate 3 mg in 3 dividing doses daily
i. Vitamin D3 10 μg in 3 dividing doses daily
j. Retinyl Acetate 1500 μg in 3 dividing doses daily

Alkylglycerols, artemisinin, agaricus bisporus and chlorophyllin form the core ingredients. Without any one of these four ingredients, the entire formulation is not likely to work effectively. Idebenone, melatonin, milk thistle, Zinc gluconate, Copper gluconate, vitamin D3 and retinyl acetate together enhance the efficacy of the formulation. Since each person's immune system is unique in some respect, these 7 ingredients allow patients with compromised, depressed or over-regulated immune systems to respond better to the formulation.

### Example 2

### Clinical Examples of the Efficacy of the Formulation

The composition was formulated as above and administered as recited, unless otherwise indicated.

5 (two males and three females) adult volunteers (ranging in age from 41-57) exhibiting chronic hepatitis infections were used for these studies. Volunteers signed comprehensive informed consent documents. Viral load was determined by PCR and checked again by immunological methods.

The presentation and results are listed in Table 1.

TABLE 1
Results.

| | | | Viral Load | Viral Load After |
|---|---|---|---|---|
| Treat- | | Presen- | Before Treat- | Treatment (Days |
| ment | Patient No. | tation | ment IU/ml | Post Treatment) |
| 1 | P103152 | HCV | 109,570 | <615 (30) |
| 2 | CS2716369 | HBV | 2,554,914 | 6,203 (84) |
| 3 | P110168 | HBV | 913 | HBsAg Negative (23) |
| 4 | 1904232289 | HCV | 92,152 | 4,192 (120) |
| 5 | 1903034858 | HCV | 5,862,749 | 3,525,005 (90) |

1) In this example, the Hepatitis C virus count dropped below 615 IU/mL in about 30 days. Before treatment, the virus count was 109,570 IU/mL.

2) In this example, the Hepatitis B virus count dropped from 2,554,914 IU/mL to 6,203 IU/mL in 84 days (dropped by 99.76% in 84 days).

3) In this example, a Chronic B carrier (HBsAg Positive) with a low virus count became a non carrier (HBsAg Negative) in 23 days after taking the mixture of supplements. (Even with very low virus counts, the HBsAg is expected to range from 300 to several thousand.)

4) In this example, the Hepatitis C virus count dropped from 92,152 IU/mL to 4,192 IU/mL in 120 days. (Dropped by 96% in 120 days.)

5) In this example, the Hepatitis C virus count dropped from 5,862,749 IU/mL to 3,525,005 IU/mL in 90 days. (Dropped by 40% in 90 days).

Although the invention has been described with reference to the above examples, it will be understood that modifications and variations are encompassed within the spirit and scope of the invention. Accordingly, the invention is limited only by the following claims.

I claim herein:

1. A dietary and/or therapeutic supplement composition consisting essentially of, in admixture:
(a) alkylglycerols;
(b) a sesquiterpene lactone;
(c) agaricus bisporous or an extract thereof; and
(d) chlorophyllin.

2. The composition of claim 1, wherein the supplement composition is formed by adding (a) to a composition comprising (b), (c) and (d) in situ or situs, and wherein the sesquiterpene lactone is artemisinin.

3. The composition of claim 2, wherein the composition comprising (b), (c) and (d) further comprises one or more ingredients selected from the group consisting of idebenone, melatonin, milk thistle, at least 2 separate gluconic acid salts, at least 2 separate fat soluble vitamins, and combinations thereof.

4. The composition of claim 3, wherein the gluconic acid salts are zinc and copper.

5. The composition of claim 3, wherein the fat soluble vitamins are vitamin D3 and retinyl acetate.

6. The composition of claim 3, comprising about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

7. The composition of claim 1, wherein the admixture comprises about 600 to 1200 mg of (a), about 600 to 1500 mg of (b), about 600 to 1000 mg of (c), and about 300 mg of (d).

8. The composition of claim 1, further comprising at least one pharmaceutically acceptable excipient.

9. The composition of claim 8, wherein the composition comprising (b), (c), and (d) is formulated in an enteric coated capsule.

10. The composition of claim 8, wherein the composition comprising (b), (c), and (d) is formulated in an enteric coated tablet.

11. The composition of claim 1, comprising about 600 to 1200 mg of (a), about 600 to 1500 mg of (b), about 600 to 1000 mg of (c), about 300 mg of (d), about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

12. A kit comprising:
i) a first container comprising an alkylglycerol containing composition;
ii) a second container comprising an artemisinin, agaricus bisporous or extract thereof, and chlorophyllin containing composition;
iii) instructions comprising procedures for mixing the compositions of the first and second container; and
iv) a label.

13. The kit of claim 12, wherein the instructions further comprise procedures for administering an admixture comprising the compositions of the first and second container to a subject in need thereof.

14. The kit of claim 12, wherein the second container further comprises one or more ingredients selected from the group consisting of idebenone, melatonin, milk thistle, at least 2 separate gluconic acid salts, at least 2 separate fat soluble vitamins, and combinations thereof.

15. The kit of claim 14, wherein the gluconic acid salts are zinc and copper.

16. The kit of claim 14, wherein the fat soluble vitamins are vitamin D3 and retinyl acetate.

17. The kit of claim 14, wherein the admixture comprises about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

18. The kit of claim 13, wherein the admixture comprises about 600 to 1200 mg of alkylglycerols, about 600 to 1500 mg of artemisinin, about 600 to 1000 mg of agaricus bisporous or extract thereof, and about 300 mg of chlorophyllin.

19. The kit of claim 12, wherein the compositions of the first and second container further comprising at least one pharmaceutically acceptable excipient.

20. The kit of claim 19, wherein the composition comprising artemisinin, agaricus bisporous or extract thereof, and chlorophyllin is formulated in an enteric coated capsule.

21. The kit of claim 19, wherein the composition comprising artemisinin, agaricus bisporous or extract thereof, and chlorophyllin is formulated in an enteric coated tablet.

22. The kit of claim 13, wherein the admixture comprises about 600 to 1200 mg of alkylglycerols, about 600 to 1500 mg of artemisinin, about 600 to 1000 mg of agaricus bisporous or extract thereof, about 300 mg of chlorophyllin, about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

23. A method of supplementing the diet of a subject comprising a step of administering to the subject the dietary supplement according to claim 1.

24. The method of claim 23, wherein when the dietary supplement is administered to said subject, the supplement composition is formed by adding (a) to a composition comprising (b), (c), and (d) in situ or situs.

25. The method of claim 23, wherein the composition of the dietary supplement comprising (b), (c), and (d) further comprises one or more ingredients selected from the group consisting of idebenone, melatonin, milk thistle, at least 2 separate gluconic acid salts, at least 2 separate fat soluble vitamins and combinations thereof, and wherein the sesquiterpene lactone is artemisinin.

26. The method of claim 23, wherein the subject is infected with a virus.

27. The method of claim 26, wherein the virus is selected from the group consisting of Hepatitis B, Hepatitis C, Epstein Barr Virus, HIV, Cytomegalovirus, Herpes Simplex Virus 1, Herpes Simplex Virus 2, Human Papillomavirus, Adenovirus, Kaposi's Sarcoma-Associated Herpesvirus, Torquetenovirus, JC Virus, and BK Virus.

28. The method of claim 24, wherein the dietary supplement is given via 3 daily doses.

29. The method of claim 28, wherein the dietary supplement administered comprises about 600 to 1200 mg of alkylglycerols, about 600 to 1500 mg of artemisinin, about 600 to 1000 mg of agaricus bisporous or extract thereof, about 300 mg of chlorophyllin, about 300 mg of idebenone, about 30 mg of melatonin, about 700 mg of milk thistle, about 30 mg of zinc gluconate, about 3 mg of copper gluconate, about 10 μg of vitamin D3, and about 1500 μg of retinyl acetate.

30. The method of claim 26, wherein administration of the dietary supplement reduces viral load.
